(19) United States
(12) Patent Application Publication
(10) Pub. No.: US 2022/0183569 A1
(43) Pub. Date: Jun. 16, 2022

(54) Blood Pressure Assessment Using Features Extracted Through Deep Learning

(71) Applicants: Anhui Huami Health Technology Co., Ltd., Hefei (CN); Zepp, Inc., Cupertino, CA (US)

(72) Inventors: Yan Vule, Port Moody (CA); Artem Galeev, Vancouver (CA); Jayanth Chennamangalam, Vancouver (CA); Kongqiao Wang, Hefei, Anhui (CN); Rana Sadeghi Chegani, Vancouver (CA)

(21) Appl. No.: 17/117,988

(22) Filed: Dec. 10, 2020

(51) Int. Cl. A61B 5/022; A61B 5/021; A61B 5/00

(52) U.S. Cl. CPC A61B 5/022; A61B 5/7264; A61B 5/02125

(57) ABSTRACT

Assessing a blood pressure of a user includes obtaining photoplethysmogram (PPG)-related signals of the user; inputting the PPG-related signals to layers of a deep-learning (DL) model, where the layers exclude an output layer; obtaining, from the layers of the DL model, features related to blood pressure; inputting to a machine-learning (ML) model the obtained features, where the ML model is different from the DL model; and obtaining, as an output of the ML model, the blood pressure of the user.

### CROSS REFERENCES TO RELATED APPLICATION(S)

NONE.

### TECHNICAL FIELD

This disclosure relates generally to assessing physiological properties of a user through sensor signals, and more specifically to detecting blood pressure using machine learning.

### BACKGROUND

Many portable devices have been developed in which sensors are used to detect variation in blood flow through arteries or blood volume in subcutaneous tissue. Applications include the monitoring of heart rate, glucose level, apnea, respiratory stress, and other physiological conditions.

High blood pressure is a major risk for heart disease. By some estimates, high blood pressure affects one in every three adults in the United States. By some other estimates, in developing and developed countries, respectively, 45% and 55% of high-blood-pressure sufferers are not aware of their condition.

The ability to monitor blood pressure via a portable (e.g., wearable) device is desirable.

### SUMMARY

Disclosed herein are implementations of a wearable device for measuring blood pressure.

A first aspect is a method for assessing a blood pressure of a user. The method includes obtaining photoplethysmogram (PPG)-related signals of the user; inputting the PPG-related signals to layers of a deep-learning (DL) model, where the layers exclude an output layer; obtaining, from the layers of the DL model, features related to blood pressure; inputting to a machine-learning (ML) model the obtained features, where the ML model is different from the DL model; and obtaining, as an output of the ML model, the blood pressure of the user.

A second aspect is a device for assessing a physiological property of a user including a sensor and a processor. The processor is configured to: acquire a signal from the sensor; obtain, using the signal, features related to the physiological property from a portion of a first machine learning (ML) model that is neural-network based ML model; and obtain, using the features related to the physiological property, an estimate of the physiological property of the user from a second ML that is not neural-network based. The first ML model is trained to estimate the physiological property. The portion does not include an output layer of the first ML model.

A third aspect is a non-transitory computer-readable storage medium that includes executable instructions that, when executed by a processor, facilitate performance of operations including obtaining sensor data; obtaining, using the sensor data, features related to a physiological property of a user that are obtained using a subset of a first machine learning (ML) model, and where the features are not the estimate of the physiological property; and obtaining the estimate of the physiological property from a second ML model using the features as input to the ML model. The second ML model is not a neural-network model.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 depicts some aspects of an illustrative implementation of an apparatus according to implementations of this disclosure.

FIG. 2 depicts some aspects of an illustrative implementation of an apparatus according to implementations of this disclosure.

FIG. 3 depicts some aspects of an illustrative implementation of an apparatus according to implementations of this disclosure.

FIGS. 4A-4B depict some aspects of a user's anatomy according to implementations of this disclosure.

FIGS. 5A-5C depict some aspects of an illustrative implementation of an apparatus according to implementations of this disclosure.

FIG. 6A-6C depict some aspects of an illustrative implementation of an apparatus according to implementations of this disclosure.

FIG. 7 depicts some aspects of an illustrative implementation of an apparatus according to implementations of this disclosure.

FIG. 8 depicts an illustrative implementation of a computing system according to implementations of this disclosure.

FIG. 9 depicts aspects of an example of using a deep learning model with a machine learning model to estimate a physiological property according to implementations of this disclosure.

FIG. 10 is an example of a diagram of a device according to implementations of this disclosure.

FIG. 11 illustrates typical signals that relate to a pulse wave and from which features can be extracted according to implementations of this disclosure.

FIG. 12 is a flowchart of an example of a technique 1200 for estimating blood pressure according to an implementation of this disclosure.

FIG. 13 is a flowchart of an example of a technique 1300 for estimating a physiological property of a user according to an implementation of this disclosure.

### DETAILED DESCRIPTION

Disclosed herein are implementations of an apparatus for sensing, measuring, analyzing, and/or displaying physiological information. In one aspect, the apparatus may be a wearable device comprising an upper module and/or a lower module. The wearable device may be worn on a user's body such that one or more sensors of the upper and lower modules contact a targeted area of tissue. In one implementation, the wearable device is a watch, band, or strap that can be worn on the wrist of a user such that the upper and lower modules are each in contact with a side of the wrist.

In an embodiment, the wearable device can be a lower module that can be attached to another device. For example, the wearable device can be a lower module that is a clip and/or an add-on to a watch or another wearable device. For example, the lower module may be attachable to the bottom of a watch such that the lower module is in contact with the skin of the wearer.

Each of the upper and lower modules may comprise one or more sensors, including but not limited to optical/PPG sensors, ECG sensors/electrodes, bio impedance sensors, galvanic skin response sensors, tonometry/contact sensors, accelerometers, pressure sensors, acoustic sensors, electro-mechanical movement sensors, and/or electromagnetic sensors. In one implementation, one or more optical/PPG sensors may comprise one or more light sources for emitting light proximate a targeted area of tissue and one or more optical detectors for detecting either reflected light (where an optical detector is located on the same side of the targeted area as the light source(s), i.e., within the same module) or transmitted light (where an optical detector is located opposite the light source(s), i.e., within an opposing module).

In a further aspect, the strap or band of the wearable device may be configured so as to facilitate proper placement of one or more sensors of the upper and/or lower modules while still affording the user a degree of comfort in wearing the device. In one implementation, rather than a strap that lies in a plane perpendicular to the longitudinal axis of the user's wrist or arm (as is the case with traditional wrist watches and fitness bands), the band may be configured to traverse the user's wrist or arm at an angle that brings one or more components of the upper or lower modules into contact with a specific targeted area of the user while allowing another portion of the band to rest at a position on the user's wrist or arm that the user finds comfortable.

In another aspect, the precise location of the upper and/or lower modules can be customized such that one or more sensors of either module can be placed in an ideal location of a user, despite the physiological differences between body types from user to user.

The aforementioned features result in more comfortable wearable device while also increasing reliability and accuracy of the device sensing, measuring, analyzing, and displaying of physiological information.

In one implementation, the physiological information sensed, measured, analyzed, or displayed can include but is not limited to heart rate information, ECG waveforms, calorie expenditure, step count, speed, blood pressure, oxygen levels, pulse signal features, cardiac output, stroke volume, and respiration rate. In further implementations, the physiological information may be any information associated with a physiological parameter derived from information received by one or more sensors of the wearable device. Regardless, the physiological information may be used in the context of, for example, health and wellness monitoring, athletic training, physical rehabilitation, and patient monitoring. Of course, these examples are only illustrative of the possibilities and the device described herein may be used in any suitable context.

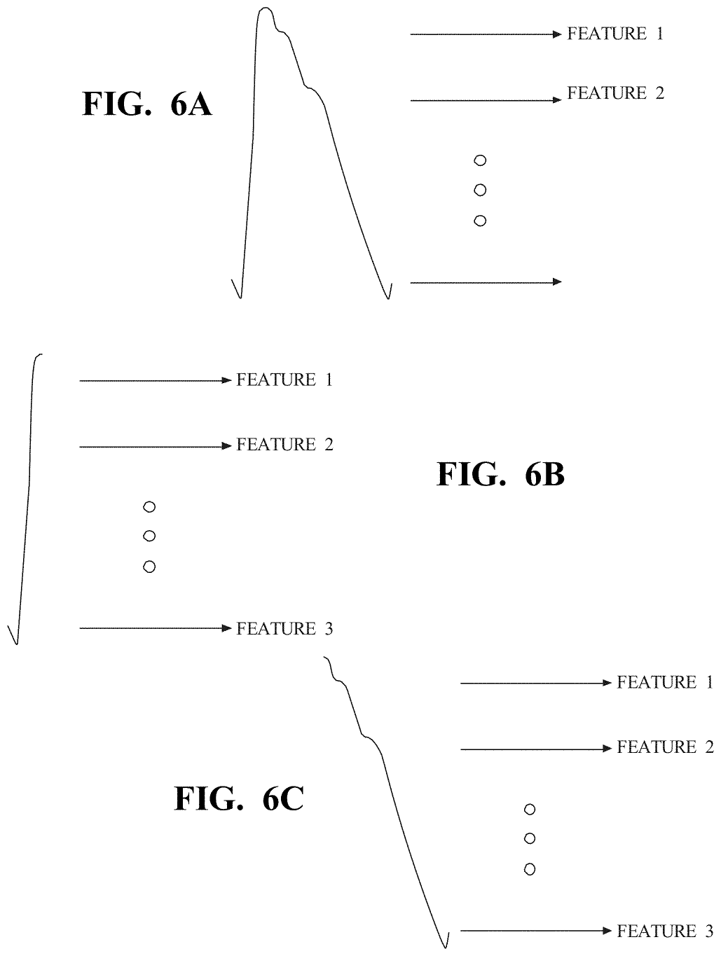

Estimation of physiological properties (such as the blood pressure (BP)) from photoplethysmograms (PPG) and/or other sensor data is typically performed using a machine learning (ML) model in one of two ways: (a) with handcrafted morphological features that are input to the machine learning (ML) to output the BP, or (b) using deep learning (DL) that may receive a raw PPG pulse as input and output the BP. Examples of handcrafted features include the height of the pulse, the width of the pulse, and other features as those described with respect to FIGS. 6A-6C and which are extracted using signal processing techniques.

A problem with handcrafted features may be that the performance of the model depends on human knowledge about the input signal. Hand-crafted features are features designed by an expert (e.g., a human) based on the expert's domain knowledge. To illustrate, in the domain of heart rate determination, an expert may know that the width of a pulse is correlated to the heart rate: a wider pulse is correlated with a slow/low heart rate; and a narrower pulse is correlated with a high heart rate (e.g., a faster beating heart). Thus, the expert would design an algorithm to extract (e.g., derive, calculate, obtain, etc.) the pulse width from sensor data, which is then used (along with other features) to determine the heart rate. Said in a more general way, the hand-crafted features are typically interpretable: the extracted features are correlated with the physiological phenomenon they are designed for.

A problem with using DL is that deep learning models may overfit to the training and result in poor generalization properties and poor prediction/inference capabilities.

Thus, aspects of this disclosure can overcome both of these problems. Implementations according to this disclosure estimate a physiological property (such as the BP) of a user using a two-step process. In a first step, features are extracted (e.g., inferred, obtained, derived, etc.) using deep learning or some other neural-network model (collectively, DL model). Thus, the DL model is merely used for feature extraction; it is not used as an actual predictor of the physiological property (e.g., the BP). In a second step, the extracted features from the DL model are used as input to a ML model, which outputs the BP.

The ML model of the second step can be, or can use, any traditional (i.e., non-neural network based) regression or classification technique, other than one that suffers from overfitting. As such, the ML model can be a gradient boosting model, an adaptive boosting model, a random forest model, a support vector machine, or some other regression or classification technique that is not a neural network and, thus, may not suffer from overfitting. Instead of (or in addition to) using handcrafted features, the ML model uses features extracted by a DL model.

While the systems and devices described herein may be depicted as wrist worn devices, one skilled in the art will appreciate that the systems and methods described below can be implemented in other contexts, including the sensing, measuring, analyzing, and display of physiological data gathered from a device worn at any suitable portion of a user's body, including but not limited to, other portions of the arm, other extremities, the head, and/or the chest.

Reference will now be made in detail to certain illustrative implementations, examples of which are illustrated in the accompanying drawings. Wherever possible, the same reference numbers will be used throughout the drawings to refer to the same or like items.

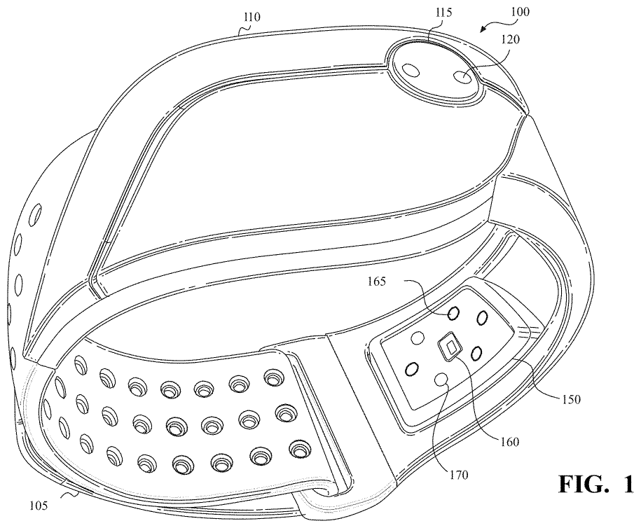

FIG. 1 depicts an illustrative implementation of an apparatus 100 according to implementations of this disclosure. In one aspect, apparatus 100 may be a physiological monitor worn by a user to sense, collect, monitor, analyze, and/or display information pertaining to one or more physiological parameters. In the depicted implementation, apparatus 100 may comprise a band, strap, or wrist watch. In further implementations, apparatus 100 may be any wearable monitor device configured for positioning at a user's wrist, arm, another extremity of the user, or some other area of the user's body.

In another aspect, apparatus 100 may comprise at least one of an upper module 110 or a lower module 150, each comprising one or more components and/or sensors for detecting, collecting, processing, and displaying one or more physiological parameters of a user and/or other information that may or may not be related to health, wellness, exercise, or physical training sessions.

In addition to upper module 110 and lower module 150, apparatus 100 may comprise a strap or band 105 extending from opposite edges of each module for securing apparatus 100 to the user. In one implementation, band(s) 105 may comprise an elastomeric material. In alternative implementations, band(s) 105 may comprise some other suitable material, including but not limited to, a fabric or metal material.

Upper module 110 or lower module 150 may also comprise a display unit (not shown) for communicating information to the user. The display unit may be an LED indicator comprising a plurality of LEDs, each a different color. The LED indicator can be configured to illuminate in different colors depending on the information being conveyed. For example, where apparatus 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees apparatus 100 through his or her peripheral vision.

In addition, or alternatively, the display unit may comprise a display screen for displaying images or characters to the user. The display unit may further comprise one or more hard or soft buttons or switches configured to accept input by the user.

Apparatus 100 may further comprise one or more communication modules. In some examples, each of upper module 110 and lower module 150 comprise a communication module such that information received at either module can be shared with the other module.

One or more communication modules can also be configured to communicate with other devices such as the user's cell phone, tablet, or computer. Communications between the upper and lower modules can be transmitted from one module to the other wirelessly (e.g., via Bluetooth, RF signal, WiFi, etc.) or through one or more electrical connections embedded in band 105. In a further implementation, any analog information collected or analyzed by either module can be translated to digital information for reducing the size of information transfers between modules. Similarly, communications between either module and another user device can be transmitted wirelessly or through a wired connection, and translated from analog to digital information to reduce the size of data transmissions.

As shown in FIG. 1, lower module 150 can comprise an array of sensors 155 including but not limited to one or more optical detectors 160, one or more light sources 165, and one or more contact pressure/tonometry sensors 170. These sensors are only illustrative of the possibilities, however, and lower module may comprise additional or alternative sensors such as one or more acoustic sensors, electromagnetic sensors, ECG electrodes, bio impedance sensors, galvanic skin response sensors, and/or accelerometers. Though not depicted in the view shown in FIG. 1, upper module 110 may also comprise one or more such sensors and components on its inside surface, i.e. the surface in contact with the user's tissue or targeted area.

In some implementations, the location of sensor array 155 or the location of one or more sensor components of sensor array 155 with respect to the user's tissue may be customized to account for differences in body type across a group of users. For example, band 105 may comprise an aperture or channel 175 within which lower module 150 is movably retained. In one implementation, lower module 150 and channel 175 can be configured to allow lower module 150 to slide along the length of channel 175 using, for example, a ridge and groove interface between the two components. In this manner, and as described in more detail below, where the user desires to place one more components of sensor array 155 at a particular location on his or her wrist, lower module 150 can be slid into the desired location along band 105. Though not depicted in FIG. 1, band 105 and upper module 110 can be similarly configured to allow for flexible or customized placement of one or more sensor components of upper module 110 with respect to the user's wrist or targeted tissue area.

In addition to the sensors and components proximate or in contact with the user's tissue, upper module 110 and/or lower module 150 may comprise additional sensors or components on their respective outer surfaces, i.e. the surfaces facing outward or away from the user's tissue. In the implementation depicted in FIG. 1, upper module 110 comprises one such outward facing sensor array 115. In one implementation, sensor array 115 may comprise one or more ECG electrodes 120. Similar to the sensor arrays of the upper and lower modules proximate or in contact with the user's tissue, outward facing sensor array 115 may further comprise one or more contact pressure/tonometry sensors, photo detectors, light sources, acoustic sensors, electromagnetic sensors, bio impedance sensors, galvanic skin response sensors, and/or accelerometers.

The outward facing sensors of sensor array 115 can be configured for activation when touched by the user (with his or her other hand) and used to collect additional information. For example, where lower module 150 comprises one or more optical detectors 160 and light sources 165 for collecting PPG and heart rate information of the user, outward facing sensor array 115 of upper module 110 may comprise ECG electrodes 120 that can be activated when the user places a fingertip in contact with the electrodes. While the optical detectors 160 and light sources 165 of lower module 150 can be used to continuously monitor blood flow of the user, outward facing sensor array 115 of upper module 110 can be used periodically or intermittently to collect potentially more accurate blood flow information which can be used to supplement or calibrate the measurements collected and analyzed by inward facing sensor array 155 of lower module 150.

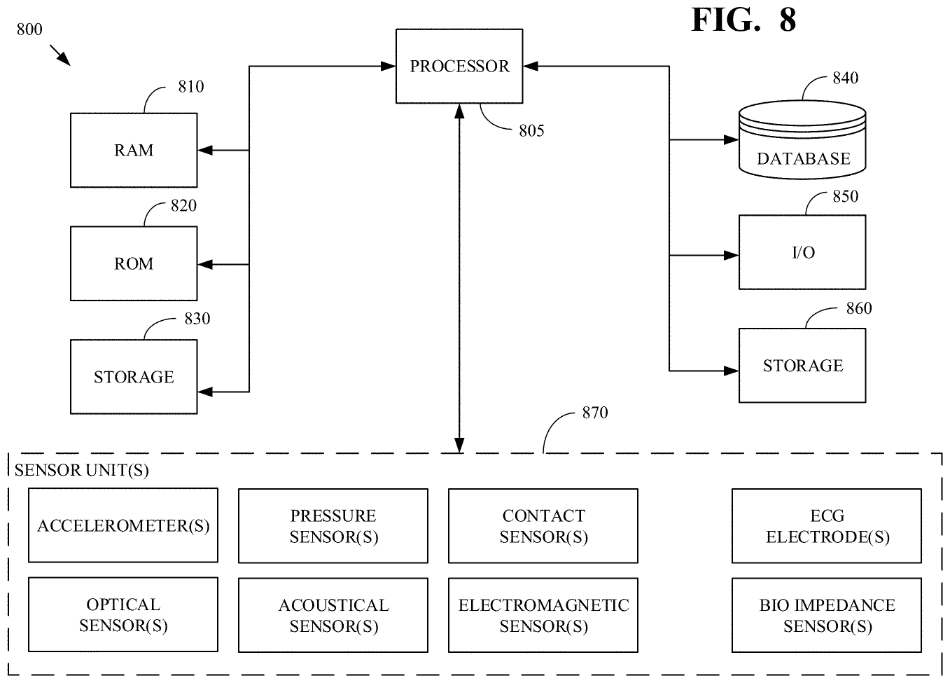

In addition to the sensor components described above with respect to each module, each module may further comprise other components for receiving, storing, analyzing, and/or transmitting physiological information. Some of those components are described below with respect to FIG. 8.

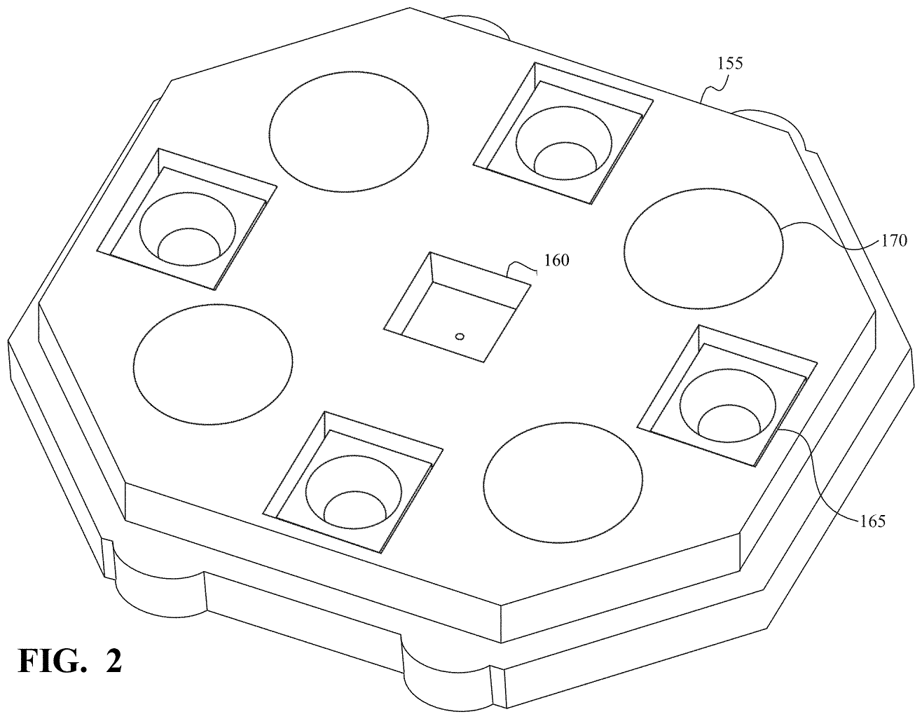

FIG. 2 depicts one implementation of inward facing sensor array 155 of lower module 150 according to implementations of this disclosure. As shown, sensor array 155 can comprise sensors including but not limited to one or more optical detectors 160, one or more light sources 165, and one or more contact pressure/tonometry sensors 170. These sensors are only illustrative of the possibilities, however, and sensor array 155 may comprise additional or alternative sensors such as one or more acoustic sensors, electromagnetic sensors, ECG electrodes, bio impedance sensors, galvanic skin response sensors, and/or accelerometers. Upper module 110 may comprise a similar inward facing sensor array (not depicted in FIG. 1) configured to position sensors proximate or in contact with the outside portion of a user's wrist or arm. In some implementations, sensor components of the upper and lower modules 110, 150 can be used in combination to collect and analyze physiological information. For example, and as described in more detail below, one or more light sources of lower module 150 can be used to transmit light through a targeted area of the user's tissue (e.g., a portion of the user's wrist) and the transmitted light can be detected by one or more photodetectors of an inward facing sensor array of upper module 110. In such an implementation, opposing modules 110 and 150 can be used to detect and analyze either reflected or transmitted light.

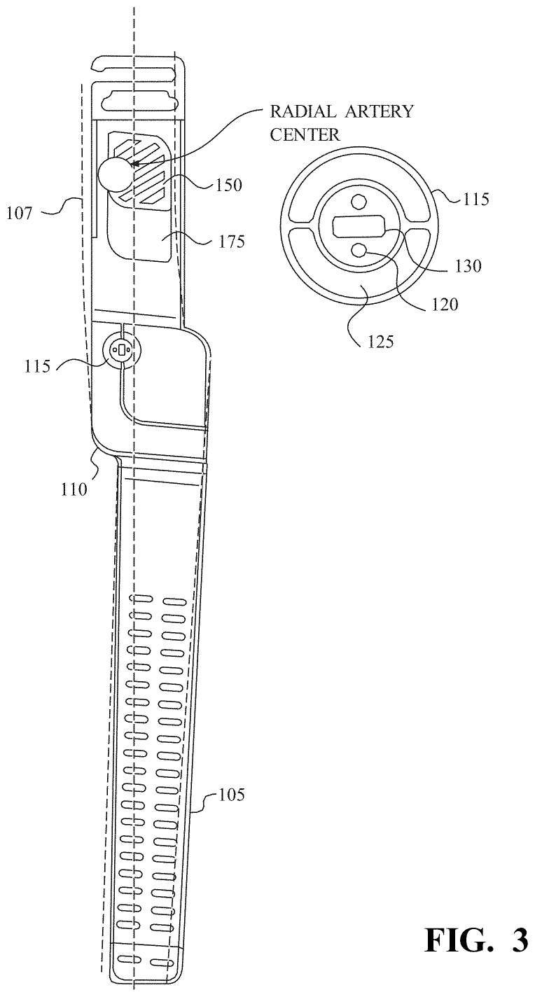

FIG. 3 depicts another view of apparatus 100 comprising band 105, upper module 110, and lower module 150 according to implementations of this disclosure. As described above, lower module 150 can be placed within channel 175 of band 105 such that lower module 150 can slide along the longitudinal axis of band 105. The movability of lower module 150 (or upper module 110 in alternative implementations) with respect to band 105 allows a user to customize the location of the inward facing sensors of lower module 150 with respect to a targeted tissue area to ensure reliable and accurate detection of physiological parameters. For example, a user can ensure that the inward facing sensors of lower module 150 are place in a location proximate the center of the user's radial artery.

In another aspect, band 105 may not extend around the user's wrist such that it traverses a circumferential path lying in a plane perpendicular to the longitudinal axis of the user's wrist or arm. Rather, the longitudinal axis of band 105 extends at an angle such that portions of inward facing sensor arrays of upper or lower modules 110, 150 can be placed at suitable locations proximate a desired targeted area of tissue while a portion of band 105 is in contact with portions of the user's wrist or arm that the user finds comfortable (i.e., above or below the wrist joint). In some implementations, where a circumferential path around a user's wrist resides in a plane perpendicular to the longitudinal extension of the user's arm or wrist, band 105 may be set at an angle 107 with respect to the perpendicular plane. In some implementations, angle 107 may be between 5° and 15° with respect to the perpendicular plane. In other implementations, angle 107 may be less than 5° or more than 15°. Of primary importance is the placement of one or more components of the sensor arrays of upper and lower modules 110, 150 proximate or in contact with a desired targeted area of tissue while allowing a portion of band 105 to be worn at a comfortable location off the user's wrist joint. Additional details regarding proper or desirable placement of one or more sensors with respect to targeted tissue areas of a user are described below with respect to other figures.

FIG. 3 also shows a closer view of outward facing sensor array 115. In the implementation depicted, sensor array 115 may comprise one or more ECG electrodes 120 for establishing an electrical connection with a user's fingertip and collected ECG data. Sensor array 115 may further comprise one or more contact pressure/tonometry sensors 125 for detecting the presence of the user's fingertip, which can trigger activation of the ECG electrodes 120. Sensor array 115 may also comprise additional or alternative components 130 such as one or more optical detectors, light sources, acoustic sensors, electromagnetic sensors, bio impedance sensors, galvanic skin response sensors, and/or accelerometers.

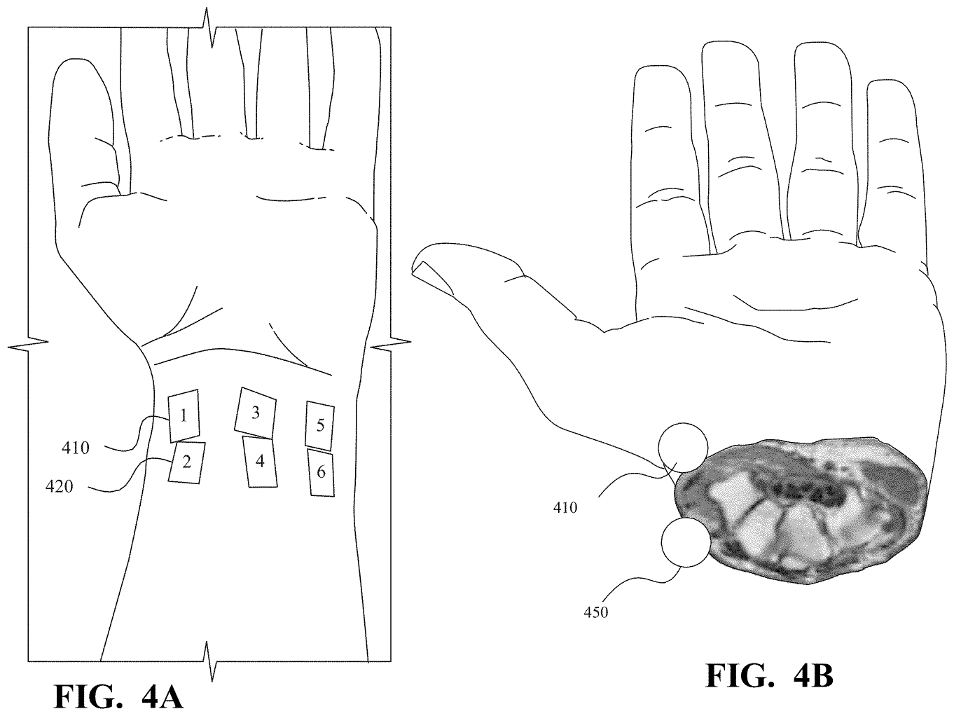

FIG. 4A depicts some points of interest on a human wrist. The best point on the wrist for detecting blood flow, for example in calculating heart rate, blood pressure, respiratory rate, etc., is at a location coinciding with the wrist joint, approximately at location 1 shown as item 410. This location is proximate the radial artery and is referred to as the CUN location.

Thus, the ideal location for a user to wear a wrist worn device is along the line across a line comprising locations 1-3-5. However, for comfort, most users prefer to wear straps or bands off the wrist joint, for example, across locations 2-4-6 shown as item 420. The result is that in most cases, users wear their monitors and corresponding sensors at a location on their wrist or arm that is not ideal and likely to introduce errors in the detection of physiological parameters.

The angle 107 of the band described with respect to FIG. 3 cures this deficiency in that it allows one or more sensor components of lower module 150 to be located above the CUN location while allowing a portion of the remaining band and/or upper module 110 to be positioned at a more comfortable location on the user's wrist or arm, such as line 2-4-6 (item 420).

Further ensuring that one or more sensors of lower module 150 can be placed at a desirable location above the CUN location, and as described in more detail above with respect to FIGS. 1 and 3, lower module 150 can slide along band 105. This allows the user to make further adjustments to the location of one or more sensors, not just along the longitudinal extension of the user's arm when apparatus 100 is in use, but also along the circumferential extension of band 105 while apparatus 100 is in use. Thus, the combination of band 105 extending around the user's wrist at an angle 107 together with the ability to slide the lower module 150 along band 105, ensure the sensors of lower module 150 can be placed at an ideal location with respect to each user (even users of different body types and physical attributes) and that the physiological parameters detected and analyzed by apparatus 100 are collected as accurately as possible.

Not only is apparatus 100 configured so as to ensure proper placement of one or more sensors and comfortability of band 105, but it also may contain additional sensors, such as a pressure sensor, at locations of apparatus 100 other than upper and lower modules 110, 150.

For example, apparatus 100 may comprise a pressure sensor located somewhere else along band 105 or at a latch that secures opposing ends of band 105 around a user's wrist for detecting pressure. Such a sensor can be used to ensure that the user is wearing the apparatus 100 tightly enough to ensure one more other sensors are in sufficient contact with a targeted area of the user's tissue to collect accurate physiological information. In alternative implementations, one or more pressure sensors of the upper and/or lower modules 110, 150 can be used to make the same determination. In either case, apparatus 100 may also be configured to alert the user (for example, via the display unit of upper module 110) if apparatus 100 is being worn too loosely or too tightly to ensure accurate measurements.

FIG. 4B depicts one example of desirable locations for one or more sensors to be placed with respect to a user's wrist or other targeted area according to implementations of this disclosure. In one implementation, one or more sensors of lower module 150 can be placed adjacent or proximate the item 410 (i.e., the CUN location) and one or more sensors of upper module 110 can be placed opposite the item 410 at point 450 of the user's wrist or targeted area. Such a configuration provides the aforementioned benefits associated with proper placement of sensors over the CUN location, but also allows for apparatus 100 to detect, collect, and analyze blood flow through the radial artery using either reflective or transmissive systems.

Wrist worn PPG sensors currently use a reflective system whereby a sensor array comprises one or more light sources and one or more optical detectors, located near one another and on the same side of a user's targeted area. The one or more light sources of the sensor array illuminate a portion of the user's tissue and light is reflected back to the optical detector(s) of the sensor array. The reflected light detected by the optical detector can be analyzed to estimate physiological parameters such as blood flow and pulse rate.

However, reflective systems may not be as accurate as transmissive systems that place one or more light sources on one side of a user's body and optical detectors on an opposing side of the user's body. One example of a transmissive system are fingertip monitors used in a clinical setting. The monitors are clipped to a patient's fingertip, one side comprising a light source for illuminating the top or bottom of the patient's fingertip, the other side comprising an optical detector for detecting the light transmitted through the fingertip.

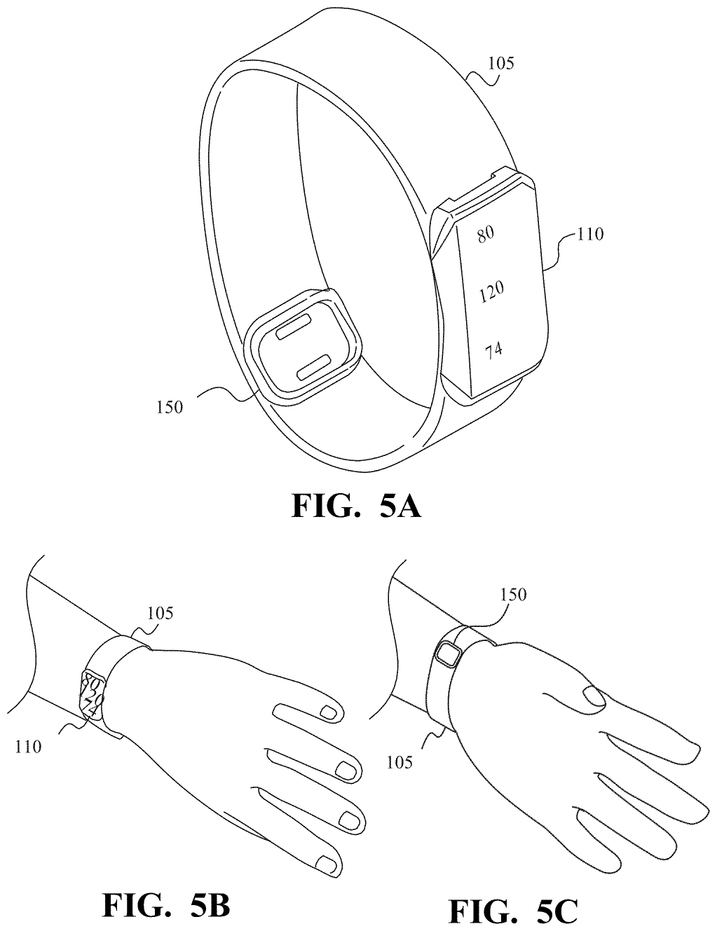

It has been thought that transmissive systems are not practical for wrist worn health monitors (or monitors worn at other locations on a user's arm or body) because the wrist is too thick for light that enters one side of a targeted area to be transmitted all the way through to the other side. However, apparatus 100 solves this problem by taking advantage of the natural location of the CUN location (the location of the radial artery at the wrist) at the inside of the wrist just under the thumb. As shown in FIGS. 5A, 5B, and 5C, apparatus 100 can be configured to place the lower module 150 comprising a light source (and/or optical detector) at the location of the CUN artery on the underside of the wrist and place the upper module 110 comprising an opposing optical detector (or light source) at a location opposite the sensors of the lower module at the periphery of the outside of the wrist just below the thumb. In this manner, the path of light transmitted through the wrist between the sensors of the lower and upper modules travels a shorter distance (shown in FIG. 4B) than if the sensors were located closer to the center of the inside and outside of a user's wrist. As a result, light illuminated from either the upper or lower module can be detected at the opposing module in a manner previously only available in clinical settings and limited to locations on the body such as the fingertip.

As described above, apparatus 100 may comprise a number of components and sensors for detecting physiological information and extracting data from it, such as blood flow, heart rate, respiratory rate, blood pressure, steps, calorie expenditure, and sleep.

Data collected from at least one or more of ECG electrodes/sensors, bio impedance sensors, galvanic skin response sensors, tonometry/contact sensors, accelerometers, pressure sensors, acoustic sensors, and electromagnetic sensors can be used for determining physiological information.

One method for determining the heart rate, respiratory rate, blood pressure, oxygen levels, and other parameters of a user involves collecting a signal indicative of blood flow pulses from a targeted area of the user's tissue. As described above, this information can be collected using, for example, a light source and a photo detector. Some implementations may use multiple light sources and they may be of varying colors (e.g., green, blue, red, etc.). For example, one light source may be an IR light source and another might be an LED light (such as a red LED). Using both an IR light source and a colored LED light (such as red) can improve accuracy as red light is visible and most effective for use on the surface of the skin while IR light is invisible yet effective for penetration into the skin. Such implementations may comprise multiple photo detectors, one or more configured to detect colored LED light (such as red) and one or more configured to detect IR light. These photo detectors (for detecting light of different wavelengths) can be combined into a single photodiode or maintained separate from one another. Further, the one or more light sources and one or more photodetectors could reside in the same module (upper or lower) in the case of a reflective system or the light source(s) could reside in one module while the optical detector(s) reside in the other in the case of a transmissive system.

Upon collection of a blood flow pulse signal, a number of parameters can be extracted from both single pulses and a waveform comprising multiple pulses. FIG. 6A depicts a single pulse from which a number of features or parameters can be extracted. Features or parameters extracted from a single pulse can include, but are not limited to, shape of the pulse, a maximum amplitude, a minimum amplitude, a maximum derivative, a time difference between main and secondary peaks, and integral through the entire extraction time (i.e., the area under the pulse). FIGS. 6B and 6C illustrate that even portions of a single pulse can be analyzed for feature extraction. Extracting features at this level of detail has a number of advantages, including the ability to capture a great number of pulse features and store each of those features digitally without having to retain the analog waveform. The result is a savings in storage requirements and ease of data transmission.

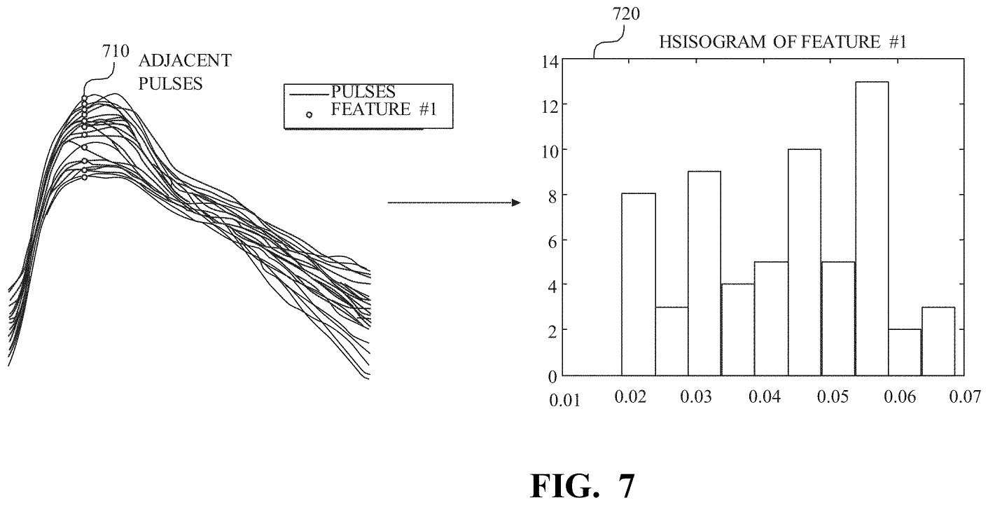

Feature extraction can also be performed on a number of pulses or a “pulse train.” FIG. 7 depicts a series of pulses overlaid with one another to show the variation among the group with respect to an identified feature according to implementations of this disclosure. In this manner, the total variation among a series of pulses with respect to a single feature can be determined. The average of a group of pulses with respect to a single feature and the standard deviation of the group with respect to the feature can also be determined. Of course, these are just examples of the types of information that can be collected from a comparison of a single feature over a group of pulses. Moreover, while FIG. 7 depicts the extraction of a single feature from the group of pulses, it should be appreciated that any number of features can be extracted from the group in a manner similar to that described above with respect to a single pulse. FIG. 7 further depicts how information collected about a single feature over a group of pulses can be digitized or presented in a histogram 720.

All of the features or parameters described above, collected using a PPG system comprising one or more light sources and/or one or more optical detectors, can be supplemented with additional sensors such as ECG electrodes/sensors, bio impedance sensors, galvanic skin response sensors, tonometry/contact sensors, accelerometers, pressure sensors, acoustic sensors, and electromagnetic sensors. For example, one or more tonometry/contact sensors can be used to extract tonometry information by measuring the contact vessel pressure. In another example, one or more acoustic sensors comprising a speaker-microphone combination (such as a micro-electro-mechanical system (“MEMS”) acoustic sensor) can be used to extract reflected sound pulses from moving vessel walls. Similarly, one or more electromagnetic sensor MEMS can be used to extract voltage induced by coils or magnet pieces pressed to moving vessel walls. In a further implementation, as described above, external or outward facing sensors can be configured to activate when touched by the off-hand (i.e., the hand on which apparatus 100 is not being worn) to collect additional information to help supplement or calibrate the information collected by the inward facing sensors of the upper or lower modules. For example, where internal facing PPG components (i.e., one or more light sources and one or more photo detectors) are used to detect reflected or transmitted light representative of blood flow pulses and some extrapolation of the data is made to determine, for example, heart rate, the user can place a fingertip of his or her off-hand on an outward facing ECG electrode (such as that shown in FIG. 1) to collect a more precise heart rate measurement. The more precise, though of more finite duration, heart rate measurement can be used to aid in the interpretation of the continuous heart rate measurements collected by the inward facing PPG sensors. The outward facing sensor can also comprise other sensors previously described herein, such as one or more contact/tonometry sensors, one or more bio impedance sensors, and one or more galvanic skin response sensors for analyzing electric pulse response. All of the information collected by an outward facing sensor from, for example, the fingertip of the user's off-hand, can be used to refine the analysis of the continuous measurements taken by any one or more of the inward facing sensors.

In addition to the inward and outward facing sensors, apparatus 100 may further comprise additional internal components such as one or more accelerometers and/or gyroscopic components for determining whether and to what extent the user is in motion (i.e., whether the user is walking, jogging, running, swimming, sitting, or sleeping). Information collected by the accelerometer(s) and/or gyroscopic components can also be used to calculate the number of steps a user has taken over a period of time. This activity information can also be used in conjunction with physiological information collected by other sensors (such as heart rate, respiration rate, blood pressure, etc.) to determine a user's caloric expenditure and other relevant information.

To determine a user's blood pressure, the PPG information described above may be combined with other sensors and techniques described herein. In one implementation, determining a user's blood pressure can comprise collecting a heart rate signal using a PPG system (i.e., one or more light sources and photo detectors) and performing feature extraction (described above) on single pulses and a series of pulses. The features extracted from single pulses and series of pulses can include statistical averages of various features across a series, information regarding the morphological shape of each pulse, the average and standard deviation of morphology of a series of pulses, temporal features such as the timing of various features within single pulses, the duration of a single pulse, as well as the average and standard deviation of the timing of a feature or duration of pulses within a series of pulses, and the timing of morphological features across a series of pulses (i.e., the frequency with which a particular pulse shape occurs in a series).

As described above, this feature extraction can not only be performed on a series of pulses and single pulses, but also on portions of a single pulse. In this manner, information pertaining to both systolic and diastolic blood pressure can be ascertained as one or more portions of an individual pulse correspond to the heart's diastole (relaxation) phase and one or more other portions of an individual pulse correspond to the heart's systole (contraction) phase. In some implementations, up to 200 features can be extracted from a partial pulse, a single pulse, and/or a series of pulses. In alternative implementations, fewer or more features may be extracted.

In addition to features extracted from PPG or ECG information, information and features can also be collected by contract/tonometry sensors, pressure sensors, bio impedance sensors galvanic skin response sensors, accelerometers, acoustic sensors, and electromagnetic sensors. For example, pressure sensors or bio impedance sensors can be used to identify blood flow pulses of user and, similar to PPG or ECG data, features can be extracted from the collected data.

The extracted features can then be cross-referenced or compared to entries in a library containing data corresponding to a population of subjects. For each subject, the library may contain information associated with each extracted feature. The library can also contain a direct measured or verified blood pressure for each subject. In further implementations, the library may contain more than one directly measured or verified blood pressure measurement for each subject, each corresponding to a subject in a different condition, such as one corresponding to the subject at rest, one corresponding to the subject engaged in light activity, and one corresponding to the subject engaged in strenuous activity. Thus, the extracted features of the user, as well as activity information pertaining to the user, can be compared to entries in the library to find one or more subjects with which the user's extracted features most closely match and the user's blood pressure can then be estimated based on the verified blood pressure of those subjects.

As one example, when features are extracted from a series of pulses, a standard deviation or range of variation across the series can be ascertained. Generally speaking, a large variation across a series of pulses can be associated with flexible, healthy veins. As a result, individuals exhibiting large pulse-to-pulse variations across a series of pulses typically have relatively low blood pressure. Conversely, little to no variation in features across a series of pulses is typically associated with relatively high blood pressure.

The library described above can be generated by extracting the same features from partial pulses, individual pulses, and series of pulses across hundreds or thousands of subjects. The subjects' verified blood pressure can also be measured such that it can be associated with each feature extracted from the subject's pulse information. The subject entries in the library can also be sorted based on information helpful for estimating blood pressure. For example, subjects in the library can be identified as male or female, belonging to a particular age group, or associated with one or more past health conditions. Individual subjects can be associated with information indicative of the subject's sex, age, weight, race, and any other medically meaningful distinction. Moreover, entries can be associated with information collected by other sensors at the time the verified blood pressure measurement was taken, including information collected by contract/tonometry sensors, pressure sensors, bio impedance sensors galvanic skin response sensors, accelerometers, acoustic sensors, and electromagnetic sensors. As just one example, if a user is determined to be engaged in physical activity (through a combination of accelerometer and heart rate data, as an example), his or her extracted features may only be compared to data in the library corresponding to subjects engaged in similar physical activity. Information pertaining to subjects contained in the library may also be correlated to each subject's resting heart rate, BMI, or some other medically significant indicia. For example, if a user is a young female with a low resting heart rate who is currently engaged in moderate activity, her extracted features should be compared to subjects in the library identified as young females with low resting heart rate whose blood pressure was verified during moderate activity rather than comparing the user's extracted features to an elderly male subject with a relatively high resting heart rate and whose blood pressure was verified during strenuous activity.

When the user's extracted features are compared to features recorded in the library, apparatus 100 can also weigh the entries of subjects most closely corresponding to the user more heavily than entries of subjects associated with indicia different from that of the user. For example, if the user is a male, features extracted from male subjects may be weighed more heavily than female subjects because a particular pulse variation in men of a particular age may correspond to relatively high blood pressure whereas the same pulse variation in women of that particular age may correspond to lower blood pressure.

According to the techniques described herein, accurate blood pressure estimates for a user can be made without requiring direct blood pressure measurement of the user. However, in some implementations, the user's blood pressure estimates can be further calibrated by direct measurement of the user's blood pressure by another device and that verified blood pressure can be input into apparatus 100 to aid in future estimations of the user's blood pressure.

Calibration can also be accomplished with an outward facing ECG sensor.

While an inward facing PPG sensor can continuously or periodically collect heart rate data of a user, occasionally the user may be prompted to place a fingertip of his or her off-hand on an outward facing ECG sensor (e.g., electrodes). The inward facing sensor arrays of apparatus 100 may contain additional electrodes thereby completing an electrical circuit through the user's body and allowing a more precise pulse waveform to be collected. Feature extraction can be performed on these pulses, series of pulses, and partial pulses in the same manner as described above with respect to PPG information and used to cross-reference the library.

In still a further implementation, where apparatus 100 determines, based on its continuous or periodic monitoring of the user's blood pressure using PPG or pressure sensors, that a user's blood pressure is unusually or dangerously high or low, apparatus 100 may prompt the user to place a fingertip of his or her off-hand on an outward facing ECG electrode in order to verify the unusual or unsafe condition. If necessary, apparatus 100 can then alert the user to call for help or seek medical assistance.

As described above, the upper and/or lower modules 110, 150 can be configured to continuously collect data from a user using its inward facing sensor arrays. However, certain techniques can be employed to reduce power consumption and conserve battery life of apparatus 100. For instance, in some implementations, only one of the upper or lower modules 110, 150 may continuously collect information. In alternative implementations, neither module may be continuously active, but may wait to collect information when conditions are such that accurate readings are most likely. For example, when one or more accelerometers or gyroscopic components of apparatus 100 indicate that a user is still, at rest, or sleeping, one or more sensors of upper module 110 and/or lower module 150 may collect information from the user while artifacts resulting from physical movement are absent.

While techniques for estimating a user's blood pressure using pulse signal, pressure, impedance, and other collected and input information has been described above, it should be appreciated that similar techniques can be employed to estimate a user's oxygen levels (SvO₂), hydration, respiration rate, and heart rate variability. For example, PPG, ECG, bio impedance, and acoustic measurements taken from the user can be cross-referenced with the aforementioned library and compared to subjects most closely matching the user (e.g., sex, age, height, weight, race, resting heart rate, BMI, current activity level, and any other medically meaningful distinction. Measured or verified hydration levels of one or more subjects can then be used to estimate the hydration level of the user. A similar process can be employed to estimate the user's oxygen levels (SvO₂), respiration rate, and heart rate variability.

FIG. 8 depicts an illustrative processor-based computing system (i.e., a system 800) representative of the type of computing system that may be present in or used in conjunction with any aspect of apparatus 100 comprising electronic circuitry according to implementations of this disclosure. Each of upper or lower modules 110, 150 may comprise any one or more components of system 800. In some implementations, one module may contain one of the components of system 800 and the other module, rather than comprising a similar component, may be in wired or wireless communication with the component residing in the other first module. Alternatively, each module may comprise a similar component as compared to the other module such that it is not necessary to communication with the first module to enjoy the functionality of the component. For example, upper module 110 may comprise storage, a power source, and/or a charging port, while lower module 150 may have access to the upper module's storage and/or draw power from the power source of the upper module through a wired or wireless connection. Alternatively, each module may have its own storage and/or power source. For the sake of simplicity, the system 800 will be described herein as if it encompasses the components of upper and lower modules 110, 150 collectively, while the reader appreciates that one or more components described herein may reside only in one module or may be found in both modules.

The system 800 may be used in conjunction with any one or more of transmitting signals to and from the one or more accelerometers, sensing or detecting signals received by one or more sensors of apparatus 100, processing received signals from one or more components or sensors of apparatus 100 or a secondary device, and storing, transmitting, or displaying information. The system 800 is illustrative only and does not exclude the possibility of another processor- or controller-based system being used in or with any of the aforementioned aspects of apparatus 100.

In one aspect, system 800 may include one or more hardware and/or software components configured to execute software programs, such as software for storing, processing, and analyzing data. For example, system 800 may include one or more hardware components such as, for example, processor 805, a random access memory module (RAM) 810, a read-only memory module (ROM) 820, a storage system 830, a database 840, one or more input/output (I/O) modules 850, an interface module 860, and one or more sensor modules 870. Alternatively and/or additionally, system 800 may include one or more software components such as, for example, a computer-readable medium including computer-executable instructions for performing methods consistent with certain disclosed implementations. It is contemplated that one or more of the hardware components listed above may be implemented using software. For example, the storage system 830 may include a software partition associated with one or more other hardware components of system 800. System 800 may include additional, fewer, and/or different components than those listed above. It is understood that the components listed above are illustrative only and not intended to be limiting or exclude suitable alternatives or additional components.

Processor 805 may include one or more processors, each configured to execute instructions and process data to perform one or more functions associated with system 800. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 8, processor 805 may be communicatively coupled to RAM 810, ROM 820, the storage system 830, database 840, I/O module 850, interface module 860, and one more of the sensor modules 870. Processor 805 may be configured to execute sequences of computer program instructions to perform various processes, which will be described in detail below. The computer program instructions may be loaded into RAM for execution by processor 805.

RAM 810 and ROM 820 may each include one or more devices for storing information associated with an operation of system 800 and/or processor 805. For example, ROM 820 may include a memory device configured to access and store information associated with system 800, including information for identifying, initializing, and monitoring the operation of one or more components and subsystems of system 800. RAM 810 may include a memory device for storing data associated with one or more operations of processor 805. For example, ROM 820 may load instructions into RAM 810 for execution by processor 805.

The storage system 830 may include any type of storage device configured to store information that processor 805 may need to perform processes consistent with the disclosed implementations.

Database 840 may include one or more software and/or hardware components that cooperate to store, organize, sort, filter, and/or arrange data used by system 800 and/or processor 805. For example, database 840 may include user profile information, historical activity and user-specific information, physiological parameter information, predetermined menu/display options, and other user preferences. Alternatively, database 840 may store additional and/or different information.

I/O module 850 may include one or more components configured to communicate information with a user associated with system 800. For example, I/O module 850 may comprise one or more buttons, switches, or touchscreens to allow a user to input parameters associated with system 800. I/O module 850 may also include a display including a graphical user interface (GUI) and/or one or more light sources for outputting information to the user. I/O module 850 may also include one or more communication channels for connecting system 800 to one or more secondary or peripheral devices such as, for example, a desktop computer, a laptop, a tablet, a smart phone, a flash drive, or a printer, to allow a user to input data to or output data from system 800.

The interface module 860 may include one or more components configured to transmit and receive data via a communication network, such as the Internet, a local area network, a workstation peer-to-peer network, a direct link network, a wireless network, or any other suitable communication channel. For example, the interface module 860 may include one or more modulators, demodulators, multiplexers, demultiplexers, network communication devices, wireless devices, antennas, modems, and any other type of device configured to enable data communication via a communication network.

System 800 may further comprise one or more sensor modules 870. In one implementation, sensor modules 870 may comprise one or more of an accelerometer module, an optical sensor module, and/or an ambient light sensor module. Of course, these sensors are only illustrative of a few possibilities and sensor modules 870 may comprise alternative or additional sensor modules suitable for use in apparatus 100. It should be noted that although one or more sensor modules are described collectively as sensor modules 870, any one or more sensors or sensor modules within apparatus 100 may operate independently of any one or more other sensors or sensor modules. Moreover, in addition to collecting, transmitting, and receiving signals or information to and from sensor modules 870 at processor 805, any one or more sensors of sensor module 870 may be configured to collect, transmit, or receive signals or information to and from other components or modules of system 800, including but not limited to database 840, I/O module 850, or the interface module 860.

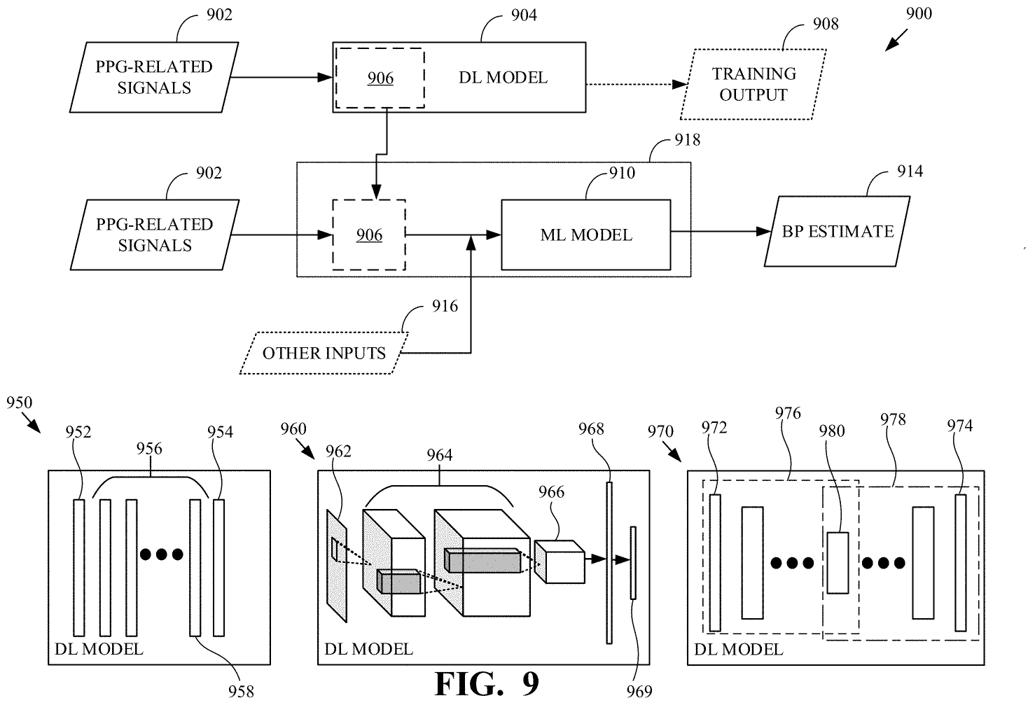

FIG. 9 depicts aspects of an example 900 of using a deep learning model with a machine learning model to estimate a physiological property according to implementations of this disclosure. The description herein uses blood pressure as the physiological property. However, the disclosure is not so limited and any physiological property can be obtained as described.

The example 900 includes a DL model 904 and a ML model 910. A BP estimate 914 is the blood pressure obtained according to implementations of this disclosure. The DL model 904 can be any type of neural network. The DL model 904 can be a regression model, such as a neural network 950 or convolutional neural network 960, which are further described below. The DL model can be an autoencoder, such as an autoencoder 970, which is further described below. As is known, machine learning models undergo a training phase before being used for inference (or classification) tasks. The training phase of the example 900 consists of first training the DL model 904 and then using the trained DL model 904 in the training of the ML model 910. The training can be supervised in the case of regression models and unsupervised in the case of autoencoders.

As mentioned, before a neural network, such as the DL model 904, can be used for an inference task (e.g., classification, regression, image reconstruction, blood pressure estimation, etc.), the neural network is trained to extract features through many layers (convolutional, recurrent, pooling, etc.), which are further described below. The neural network becomes (e.g., learns) a function that projects (e.g., maps) inputs on a latent space relevant to the task. In other words, the latent space is the space where the features lie. The latent space contains a compressed representation of the inputs. This compressed representation is then used to perform the task that the DL model is trained to perform (e.g., reconstruct an input as faithfully as possible, estimate a blood pressure, etc.). To perform well, a neural network has to learn to extract the most relevant features (e.g., the most relevant latent space).

Via training, the DL model 904 that is a regression model can learn a mapping from input 902 of the DL model 904 to a corresponding BP estimate (i.e., a training output 908). However, the training output 908, as illustrated by the dotted lines, is not the blood pressure that is estimated (e.g., obtained, inferred, etc.) according to implementations of this disclosure. To reiterate, the training BP estimate (i.e., the training output 908) is merely used during the training process. For example, the training output 908 can be used in an objective function to train the DL model 904 using backpropagation. After the DL model 904 is trained, a portion 906 of the DL model 904 is then used to train the ML model 910 and the portion 906 is then used with the ML model 910 during the inference phase. It is noted that the training output 908 can include one or both of the systolic blood pressure and the diastolic blood pressure. As already mentioned, the DL model 904 can be an autoencoder. As such, the training output 908 can be similar to the input 902. That is, the autoencoder maps from an input to itself.

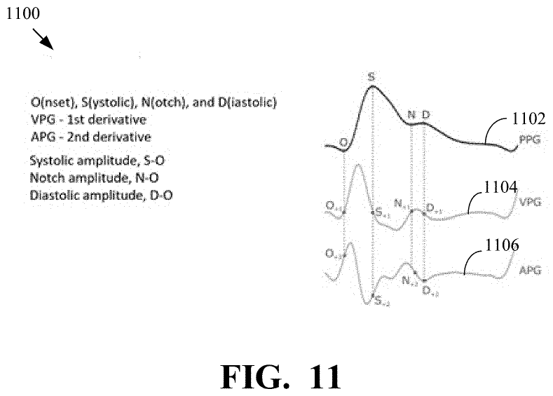

In one aspect, the input 902 can be any time-series PPG-related signals. In an example, the input 902 can be the PPG-related signal itself. That is, the input 902 can be the signal obtained from the PPG sensors. In an example, the input 902 can also include one or more transformations of the PPG signal. In an example, the transformations of the PPG signal can include a Velocity of PPG (VPG) signal, which is the first derivative of the PPG signal, and/or an Acceleration PlethysmoGram (APG) signal, which is the second derivative of the PPG signal. Other transformations (e.g., higher derivatives of the PPG signal) can be included in the input 902. The PPG, VPG, and APG signals are further described with respect to FIG. 11.

The input 902 to the DL model can be a number of amplitudes of the signals over a predetermined time window. Each window of the input can include or can represent at least one pulse (e.g., at least one heart beat). For example, raw (e.g., analog) PPG-related signals may be received or derived, the raw PPG signals can then be sampled at a certain sampling rate to produce the inputs 902.

In an example, the window can include only one pulse and the window may need to be padded. Padding may be required because the input to the DL model may be of a fixed length. To illustrate, the window length may be is 128 samples (i.e., digitized samples) and the combination of sampling rate and heart rate is such that one pulse is only 100 samples long. In this case, the window would contain 100 samples from a pulse with an additional 28 padded values. The padding value may be zero, the last sampled value, or some other value. In an example, the window can include more than one pulse and some sampled values may not be included in the window. To illustrate, the window length may be 1024 samples long, a typical pulse is 100 samples long, and every pulse is 100 samples long. In this case, each window would contain 10 full pulses and 24 samples from the 11^{th} pulse. It is noted that the window sizes, sampling rates, and the numbers of samples per pulse used in the foregoing examples are merely for illustration and may not necessarily be used in practice.

As such, the input 902 can be an array/vector of values; the values being the sampled values. For example, assuming that the inputs 902 includes PPG, VPG, and AVG values, then the sampled values within a window of the PPG, VPG, and AVG can be stacked into a 3×N array, where the 3 corresponds to each of the PPG, VPG, and AVG, and N corresponds to the number of samples.

In one aspect, the input 902 may further include signals (e.g., time series data) from other sensors, such as an accelerometer. Signals from the other sensors may be stacked onto the PPG-related signals and input into the DL model 904. In one aspect, the input 902 may further include health information of the user, whose PPG-related signals are part of the input 902.

In one example, the DL model can be a regression model, such as the neural network 950 (e.g., a fully connected neural network). The neural network 950 includes multiple layers including an input layer 952, an output layer 954, and one or more internal layers, including a layer 958. The layer 958 may be referred to herein as a layer of interest. While the layer of interest in the neural network 950 is shown as being the last layer before the output layer 954, that need not be the case. The layer of interest can be any internal layer. The output layer 954 outputs a prediction of blood pressure (i.e., the training output 908). As already mentioned, the output of the output layer 954 is only used for training of the neural network 950. Layers of the neural network 950 up to (i.e., and including) the layer 958 of interest constitute the portion 906 of the DL model 904.

In another example, the DL model can be a convolutional neural network (CNN), such as the CNN 960 and the feature-extraction portion (or a portion thereof) of the CNN 960 can constitute the portion 906 of the DL model 904. The CNN 960 is composed of an input layer 962, a number of convolutional operations 964 (e.g., the feature-extraction portion), followed by a number of fully connected layers 968, and an output layer 969. The number of operations of each type and their respective sizes is typically determined during the training phase of the machine learning. As a person skilled in the art recognizes, additional layers and/or operations can be included in each portion. For example, combinations of Pooling, MaxPooling, Dropout, Activation, Normalization, BatchNormalization, and other operations, such as a pooling layer 966, can be grouped with convolution operations (i.e., in the features-extraction portion) and/or the fully connected operation (i.e., in the classification portion). The fully connected layers may be referred to as Dense operations. As a person skilled in the art recognizes, a convolution operation can use a SeparableConvolution2D or Convolution2D operation.

A convolution layer can be a group of operations starting with a Convolution2D or SeparableConvolution2D operation followed by zero or more operations (e.g., Pooling, Dropout, Activation, Normalization, BatchNormalization, other operations, or a combination thereof), until another convolutional layer, a Dense operation, or the output of the CNN is reached. Similarly, a Dense layer can be a group of operations or layers starting with a Dense operation (i.e., a fully connected layer) followed by zero or more operations (e.g., Pooling, Dropout, Activation, Normalization, BatchNormalization, other operations, or a combination thereof) until another convolution layer, another Dense layer, or the output of the network is reached. The boundary between feature extraction based on convolutional networks and a feature classification using Dense operations can be marked by a Flatten operation, which flattens the multidimensional matrix from the feature extraction into a vector.

In a typical CNN, each of the convolution layers may consist of a set of filters. While a filter is applied to a subset of the input data at a time, the filter is applied across the full input, such as by sweeping over the input. The operations performed by this layer are typically linear/matrix multiplications. The output of the convolution filter may be further filtered using an activation function. The activation function may be a linear function or non-linear function (e.g., a sigmoid function, an arcTan function, a tanH function, a ReLu function, or the like).

Each of the fully connected operations is a linear operation in which every input is connected to every output by a weight. As such, a fully connected layer with N number of inputs and M outputs can have a total of N×M weights. As mentioned above, a Dense operation may be generally followed by a non-linear activation function to generate an output of that layer.

In the CNN 960, the feature-extraction portion includes the set of convolutional operations (e.g., the convolutional operations 964 and the pooling layer 966), which is typically a series of filters that are used to filter an input signal based on a filter. For example, and in the context of PPG signal analysis for estimating a blood pressure, these filters can be used to find features in PPG signals that are determinative of the blood pressure. The features can be used to map the features to a blood pressure. As the number of stacked convolutional operations increases, later convolutional operations can find higher-level features.

In the CNN 960, a classification portion is typically the set of fully connected layers 968, which may also be referred to as dense operations. The fully connected layers can be thought of as looking at all the input features of the PPG signals in order to generate a high-level classifier. Several stages (e.g., a series) of high-level classifiers eventually generate the desired classification or regression (such as in the case that the target is a value of physiological property) output.

In another example, the DL model 904 can be an autoencoder, such as the autoencoder 970. As is known, the autoencoder 970 consists of an encoder 976 and a decoder 978. Given an input (presented at an input layer 972) and an objective function, the autoencoder can reproduce (at an output layer 974) a compact representation of the input. The compact representation includes only those salient parts (e.g., features) of the input that are most relevant to optimizing the objective function.

Thus, when the input 902 (e.g., PPG-related signals) are input at the input layer 972, a compact and reproduced PPG-related signals are reproduced at the output layer 974. In the encoder 976, the input(s) go through a set of layers that successively reduce the dimensionality of the input. At a bottleneck layer 980, the autoencoder extracts (e.g., encodes) the essences of the signals that are most relevant to optimizing the objective function. In this case, the objective function can be to minimize a prediction of the BP and a ground truth value of the BP. The essence of the signals are the most basic features of the signals that would be used by the decoder 978 to reconstruct the original signals. The bottleneck layer 980 is a layer of interest. Thus, up to (i.e., up to and including) the bottleneck layer 980 are extracted and constitute the portion 906.

Extracting up to the layer of interest can mean extracting the parameters of all the extracted layers, including the number of layers, the number of nodes in each layer, the weights of the nodes, and other parameters necessary to reconstitute the portion 906 (i.e., the extracted layers). By the time the input has propagated to the layer of interest, the DL model 904 would have thrown away (e.g., discarded, ignored, abstracted out, etc.) at least some of the superfluous parts of the input and will have retained (e.g., remembered, learned, etc.) those features that, in the case of a regression model, are correlated with blood pressure estimation, and, in the case of an autoencoder, are correlated with morphological properties and/or physiological properties required for successful reconstruction of the original input signal.

The ML model 910 is now trained using the portion 906 of the trained DL model 904. The input 902 is fed into the portion 906. The output of the portion 906 is used as an input to the ML model 910. The ML model 910 is trained to output the BP estimate 914, which can include one or both of the systolic blood pressure and the diastolic blood pressure. As mentioned above, the ML model 910 is not a neural network. The combination of the trained portion 906 and the trained ML model 910 can be used together, as illustrated by a block 918 to estimate the BP of a user.

In some aspects, other inputs (e.g., inputs 916) can additionally be used as input to the ML model 910. In an example, handcrafted morphological features can also be used. For example, at least some of the features described with respect to FIG. 11 can be used as part of the inputs 916. In an example, features such as those described with respect to FIGS. 6A-6C can be used. In an example, personal-related information, such as sex, age, weight, race, and any other medically meaningful distinction/information (e.g., history of hypertension, etc.) can also be used. In an example, data from other sensors can also be used. For example, accelerometer data, which can be provide information related to respiration, can also be used.

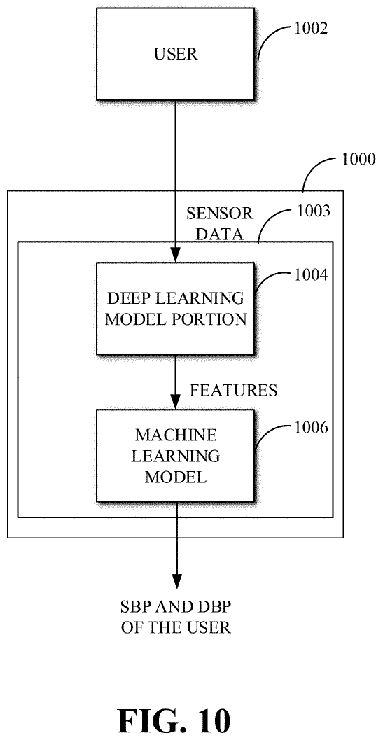

FIG. 10 is an example of a diagram of a device 1000 according to implementations of this disclosure. The device 1000 can be a wearable device and can be worn by a user 1002. The device 1000 can be the apparatus 100 of FIG. 1. The device 1000 includes sensors (not shown), which include PPG sensors. Data from the sensors of the device are acquired from the user 1002. The device 1000 can be used to estimate a physiological property, such as a blood pressure as described below, of the user.

The device 1000 can include a module 1003, which can be as illustrated with respect to the block 918 of FIG. 9, for estimating a blood pressure of the user using the data from the sensors. The sensor data can be input into a DL model portion 1004, which can be as described with respect to the portion 906 of the block 918 of FIG. 9. Features related to blood pressure of the user are extracted by the DL model portion (i.e., the portion 906) and are input to a ML model 1006, which can be the ML model 910 of FIG. 9. The ML model 1006 outputs an estimate of the blood pressure of the user, such as Systolic blood pressure (SBP) and diastolic blood pressure (DBP) of the user.

FIG. 11 illustrates PPG-related signals 1100. The PPG-related signals includes a PPG waveform 1102, a VPG signal 1104, and an APG signal 1106. One or more features can be extracted (e.g., derived, calculated, measured, etc.) from each of the PPG, VPG, and APG signals. Some of the features are described herein; however, any features are also possible.

The PPG waveform 1102 illustrates the onset 0 that is the start of the systolic phase and the peak (S) that is the end of the systolic phase. The diastolic notch is indicated by the (N). The pulse wave diastolic peak is marked by (D). The pulse wave end (PWE) (not specifically shown) is indicated by a valley at the end of the diastolic phase. A local maximum or an inflection point between the peak and the PWE marks the pulse wave diastolic peak (PWDP). The vertical amplitude distance between the onset (O) and peak (S), (i.e., S-O) is the systolic amplitude. The difference between diastolic notch (N) and the onset (O) (i.e., N-O) is the notch amplitude. The difference between the diastolic peak (D) and the onset (O) (i.e., D-O) is the diastolic amplitude. The horizontal distance between the onset (O) and the PWE is the pulse wave duration (PWD). Each of these can be used as features to be used as input to the ML model.

The VPG signal 1104 is the first derivative of the PPG and represents the velocity of blood at a point of measurement of the pulse wave (e.g., wrist, finger, etc.). The APG signal 1106 is the second derivative of the PPG signal and represents the acceleration of blood flow at the point of measurement of the pulse wave.

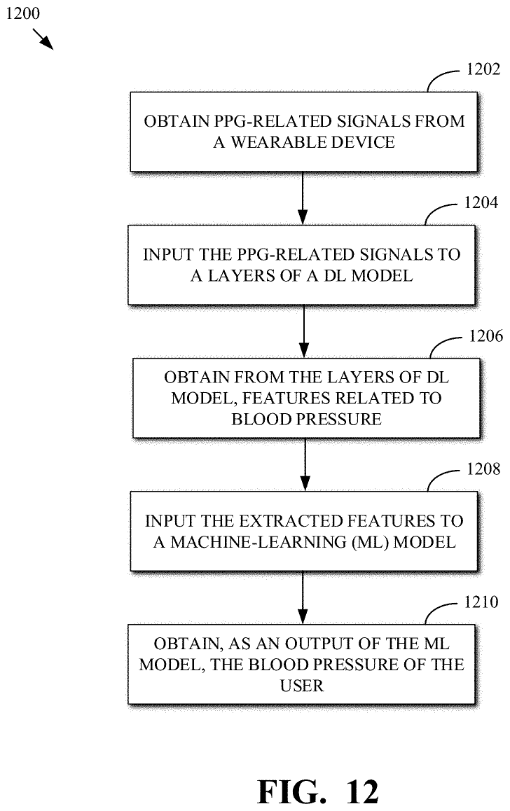

FIG. 12 is a flowchart of an example of a technique 1200 for estimating blood pressure according to an implementation of this disclosure. The technique 1200 can be implemented by a wearable device that is worn by a user, such as the apparatus 100 of FIG. 1 or the apparatus 900 of FIG. 9. The technique 1200 can be implemented as executable instructions that can be stored in a memory, such as one of the storage system 830 or the ROM 820 of FIG. 8. The instructions can be executed by a processor, such as the processor 805 of FIG. 8, to perform the steps of the technique 1200. The technique 1200 can be implemented using specialized hardware or firmware.

At 1202, the technique 1200 obtains photoplethysmogram (PPG)-related signals of the user. For example, a raw PPG signal can be acquired from PPG sensors of the wearable device. The raw PPG signal can be sampled to obtain the PPG-related signal. In an example, the PPG-related signals can also include sampled VPG and/or APG signals. In an example the PPG-related signals can also include higher derivates of the PPG signal.

At 1204, the technique 1200 inputs the PPG-related signals to layers of a deep-learning (DL) model. As described above with respect to the DL model 904 of FIG. 9, the DL model is trained to obtain the blood pressure of the user. In an example, and as also described with respect to FIG. 9, the DL model can be an autoencoder that includes an encoder and a decoder and the encoder constitutes the layers of the DL model. As such, the features can be obtained from a bottleneck layer of the autoencoder. In another example, the DL model can be a convolutional neural network (CNN) and the layers of the DL model can be the feature extraction layers of the CNN. In another example, the DL model can be a fully connected neural network and the layers of the DL can be the layers of the neural network up to a layer of interest. In an example, the layer of interest can be the last layer that is connected to the output layer. In an example, the first ML is convolutional neural network (CNN) and at least a subset of a feature-extraction portion of the CNN constitutes the portion of the first ML model.

At 1206, the technique 1200 obtains, from the layers of the DL model, features related to blood pressure. In the case of an autoencoder, the features related to blood pressure can be more strictly defined as features that are correlated with morphological properties and/or with physiological properties (such as BP). The features can be obtained as described with respect to the module 1003 of FIG. 10 or the block 918 of FIG. 9.

At 1208, the technique 1200 inputs the extracted features to a machine-learning (ML) model. As described above, the ML model is a machine learning model that is not neural-network based. In an example, and as described above with respect to the other inputs 916, additional inputs can also be used.

At 1210, the technique 1200 obtains, as an output of the ML model, the blood pressure of the user.

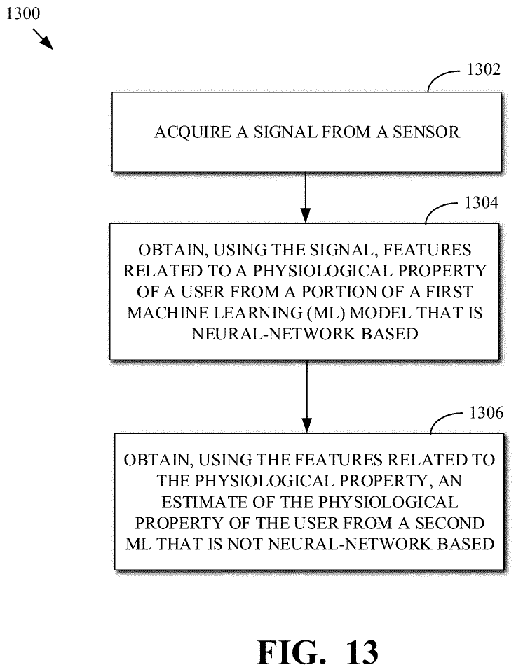

FIG. 13 is a flowchart of an example of a technique 1300 for estimating blood pressure according to an implementation of this disclosure. The technique 1300 can be implemented by a device, such as a wearable device 100 of FIG. 1. The technique 1300 can be implemented by a computer device, such as the computing device 800 of FIG. 8. The technique 1300 can be implemented as software executable instructions that may be stored in a memory such as one of the storage system 830 or the ROM 820 of FIG. 8. The instructions can be executed by a processor, such as the processor 805 of FIG. 8, to perform the steps of the technique 1300. The technique 1300 can be implemented using specialized hardware or firmware.

At 1302, the technique 1300 obtains a signal from a sensor. For example, a raw PPG signal can be acquired from PPG sensors of the device. The raw PPG signal can be sampled to obtain the PPG-related signal. In an example, the PPG-related signals can also include sampled VPG and/or APG signals. In an example the PPG-related signals can also include higher derivates of the PPG signal.

At 1304, the technique 1300 obtains, using the signal, features related to a physiological property of the user from a portion of a first machine learning (ML) model that is neural-network based. In an example, the first ML model may be the DL model 904 of FIG. 9 as described above. In an example, the neural-network-based first ML model is trained to estimate the physiological property. In an example, the first ML model is trained to reconstruct an input to the first ML model. The portion of the first ML does not include an output layer of the neural-network-based first ML model. In an example, the features related to the physiological property of the user can be obtained as described with respect to the module 1003 of FIG. 10 or the block 918 of FIG. 9.

In an example, the physiological property of the user can be a blood pressure of the user, the sensor can be a PPG sensor, and the signal can be a PPG signal. In another example, the physiological property of the user can be a pulse transit time. In an example, the technique 1300 uses the PPG signal to obtain the features related to the physiological property. In an example, the technique 1300 uses at least one of a first derivative of the PPG signal, a second derivative of the PPG signal, or a derivate higher than the second derivate to obtain the features related to the physiological property.

In an example, and as also described with respect to FIG. 9, the first ML model can be an autoencoder which may include an encoder and a decoder, and the portion of the first ML model can be the encoder of the autoencoder. In an example, the first ML model can be a fully connected neural network and the portion of the first ML model constitutes layers up to a layer of interest and the layer of interest is not an output layer. In an example, the layer of interest is a layer connected to the output layer. In an example, the first ML is convolutional neural network (CNN) and at least a subset of a feature-extraction portion of the CNN constitutes the portion of the first ML model.

At 1306, the technique 1300 obtains, using the features related to the physiological property, an estimate of the physiological property of the user from a second ML that is not neural-network based.

While implementations have been illustrated and described, it will be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for assessing a blood pressure of a user, comprising:
obtaining photoplethysmogram (PPG)-related signals of the user;
inputting the PPG-related signals to layers of a deep-learning (DL) model, wherein the layers exclude an output layer;
obtaining, from the layers of the DL model, features related to blood pressure;
inputting to a machine-learning (ML) model the obtained features, wherein the ML model is different from the DL model; and
obtaining, as an output of the ML model, the blood pressure of the user.

2. The method of claim 1, wherein the PPG-related signals comprise: photoplethysmograph signals, velocity photoplethysmogram signals (VPG), and acceleration photoplethysmogram signals (APG).

3. The method of claim 2, wherein the PPG-related signals further comprise a derivate, higher than a second derivative of the photoplethysmograph signals.

4. The method of claim 1, wherein the DL model is trained to obtain the blood pressure of the user.

5. The method of claim 1, wherein the DL model comprises an autoencoder, and the features related to the blood pressure are obtained from a bottleneck layer of the autoencoder.

6. The method of claim 5, wherein an encoder part of the DL model constitute the layers of the DL model.

7. The method of claim 1, wherein the DL model comprises fully connected layers, and the features related to the blood pressure are obtained from a fully connected layer of the DL model that that is not the output layer of the DL.

8. A device for assessing a physiological property of a user, comprising:
a sensor; and
a processor configured to:
acquire a signal from the sensor;
obtain, using the signal, features related to the physiological property from a portion of a first machine learning (ML) model that is neural-network based ML model,
wherein the first ML model is trained to estimate the physiological property, and
wherein the portion does not include an output layer of the first ML model; and
obtain, using the features related to the physiological property, an estimate of the physiological property of the user from a second ML that is not neural-network based.

9. The device of claim 8, wherein the physiological property of the user is a blood pressure of the user, the sensor is a PPG sensor, and the signal is a PPG signal.

10. The device of claim 8, wherein the physiological property of the user is a pulse transit time.

11. The device of claim 9, wherein to obtain, using the signal, the features related to the physiological property comprises to:
use the PPG signal to obtain the features related to the physiological property.

12. The device of claim 11, wherein to obtain, using the signal, the features related to the physiological property further comprises to:
use at least one of a first derivative of the PPG signal, a second derivative of the PPG signal, or a derivate higher than the second derivate of the PPG signal to obtain the features related to the physiological property.

13. The device of claim 8, wherein the first ML is an autoencoder comprising an encoder and a decoder, and the portion of the first ML model being the encoder of the autoencoder.

14. The device of claim 8, wherein the first ML model is a fully connected neural network and the portion of the first ML model constitutes layers up to a layer of interest, wherein the layer of interest is not the output layer.

15. The device of claim 14, wherein the layer of interest is a layer connected to the output layer.

16. The device of claim 8, wherein the first ML is convolutional neural network (CNN) and at least a subset of a feature-extraction portion of the CNN constitutes the portion of the first ML model.

17. A non-transitory computer-readable storage medium, comprising executable instructions that, when executed by a processor, facilitate performance of operations, comprising:
obtaining sensor data;
obtaining, using the sensor data, features related to a physiological property of a user, wherein the features are obtained using a subset of a first machine learning (ML) model, and wherein the features are not the estimate of the physiological property; and
obtaining the estimate of the physiological property from a second ML model using the features as input to the ML model, wherein the second ML model is not a neural-network model.

18. The non-transitory computer-readable storage medium of claim 17, wherein the second ML model is at least one of a gradient boosting model, an adaptive boosting model, a random forest model, or a support vector machine.

19. The non-transitory computer-readable storage medium of claim 17, wherein the first ML model is at least one of a convolutional neural network or an autoencoder.

20. The non-transitory computer-readable storage medium of claim 17, wherein the physiological property of the user is blood pressure of the user.
